(19) United States
(12) Patent Application Publication
(10) Pub. No.: US 2025/0143607 A1
(43) Pub. Date: May 8, 2025

(54) ROBUST FRACTIONAL SATURATION DETERMINATION

(71) Applicant: Masimo Corporation, Irvine, CA (US)

(72) Inventors: Philip Perea, Aliso Viejo, CA (US); Ammar Al-Ali, San Juan Capistrano, CA (US); Massi Joe E. Kiani, Laguna Niguel, CA (US)

(21) Appl. No.: 19/014,104

(22) Filed: Jan. 8, 2025

Related U.S. Application Data
(63) Continuation of application No. 17/377,170, filed on Jul. 15, 2021, now Pat. No. 12,226,206.
Application No. 17/377,170 is a continuation of application No. 16/387,352, filed on Apr. 17, 2019, now Pat. No. 11,089,982.
Application No. 16/387,352 is a continuation of application No. 15/795,007, filed on Oct. 26, 2017, now Pat. No. 10,299,709.
Application No. 15/795,007 is a continuation of application No. 13/791,633, filed on Mar. 8, 2013, now Pat. No. 9,808,188.
Application No. 13/791,633 is a continuation of application No. 13/650,730, filed on Oct. 12, 2012, now abandoned.
(60) Provisional application No. 61/703,688, filed on Sep. 20, 2012.
(60) Provisional application No. 61/547,001, filed on Oct. 13, 2011.

(51) Int. Cl. A61B 5/1455; A61B 5/00

(52) U.S. Cl. CPC A61B 5/14551; A61B 5/7203; A61B 5/7221

(57) ABSTRACT

The present disclosure describes the derivation and measurement of a fractional oxygen saturation measurement. In one embodiment, a system includes an optical sensor and a processor. The optical sensor can emit light of multiple wavelengths directed at a measurement site of tissue of a patient, detect the light after attenuation by the tissue, and produce a signal representative of the detected light after attenuation. The processor can receive the signal representative of the detected light after attenuation and determine, using the signal, a fractional oxygen saturation measurement based on two or more different measures of fractional oxygen saturation.

### PRIORITY CLAIM TO RELATED APPLICATIONS

The present application is a continuation of U.S. patent application Ser. No. 17/377,170, filed Jul. 15, 2021, which is a continuation of U.S. patent application Ser. No. 16/387,352, filed Apr. 17, 2019, now U.S. Pat. No. 11,089,982, entitled “Robust Fractional Saturation Determination,” which is a continuation of U.S. patent application Ser. No. 15/795,007, filed Oct. 26, 2017, now U.S. Pat. No. 10,299,709, entitled “Robust Fractional Saturation Determination,” which is a continuation of U.S. patent application Ser. No. 13/791,633, filed Mar. 8, 2013, now U.S. Pat. No. 9,808,188, entitled “Robust Fractional Saturation Determination,” which is a continuation-in-part of U.S. patent application Ser. No. 13/650,730, filed Oct. 12, 2012, entitled “Robust Fractional Saturation Determination,” which claims priority benefit under 35 U.S.C. § 119(e) of U.S. Provisional Application Ser. No. 61/703,688, filed Sep. 20, 2012, entitled “Robust Fractional Saturation Determination,” and U.S. Provisional Application Ser. No. 61/547,001, filed Oct. 13, 2011, entitled “Robust Fractional Saturation Determination;” the disclosures of which are incorporated herein by reference.

### FIELD OF THE DISCLOSURE

The present disclosure relates to the field of non-invasive optical based physiological measurements.

### BACKGROUND

Pulse oximeters are well known and accepted for use in clinical environments. Pulse oximeters measure the relative percentage of oxygen carrying hemoglobin molecules in the blood. This measurement is commonly referred to as oxygen saturation or SpO₂. Pulse oximeters generally shine light of predetermined wavelengths at a measurement site on the patient and measure the attenuation of the light by the tissue using a detector. Some Pulse oximeters also include additional capabilities to measure other blood analyte levels in addition to oxygen saturation. Non-invasive measurement devices capable of measuring multiple physiological parameters, including oxygen saturation, methemoglobin levels and carboxyhemoglobin levels, are available from Masimo Corporation of Irvine CA.

### SUMMARY

Oxygen saturation provides a measure of the percentage of oxygenated hemoglobin to non-oxygenated hemoglobin. However, oxygen saturation does not take into account dishemoglobins including methemoglobin or carboxyhemoglobin that may affect the actual number of total hemoglobin molecules carrying oxygen. This is a serious problem when, for example, a patient with elevated dishemoglobins measures a high oxygen saturation measurement. Although the oxygen saturation measurement may be high, the patient may be in need of oxygen therapy because the patient's total capacity to carry oxygen is lowered by the effects of the dishemogobins. Thus, despite a high oxygen saturation measurement, the patient may need additional oxygen.

Embodiments of the present disclosure provide a measurement of fractional oxygen saturation that takes into account dishemoglobins, such as, for example, carboxyhemoglobin and methemoglobin and provides a care giver with a more accurate picture of the patient's blood analyte.

### BRIEF DESCRIPTION OF THE DRAWINGS

FIG. 1 illustrates an embodiment of a physiological measurement device according to an embodiment.

FIG. 2 illustrates a schematic diagram of an embodiment of a physiological measurement device according to an embodiment.

FIG. 3 illustrates a chart for weighting a carboxyhemoglobin measurement when methemoglobin is low.

FIGS. 4A-C illustrate flowcharts detailing rules for determining carboxyhemglobin measurements in view of methemoglobin measurements.

FIG. 5 illustrates a fractional oxygen saturation determination system for determining fraction oxygen saturation from a plurality of different fraction oxygen saturation measurements.

### DETAILED DESCRIPTION

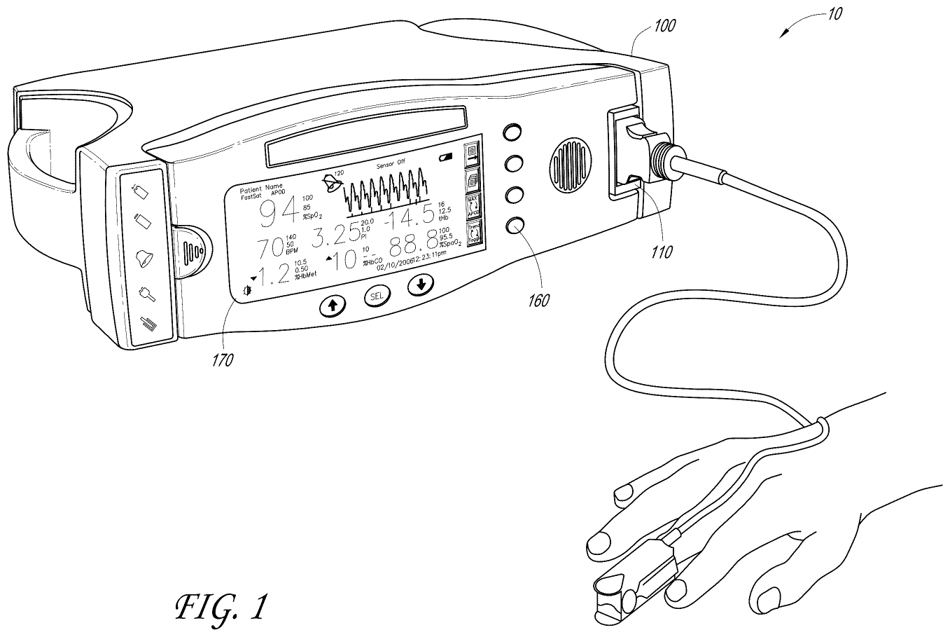

FIG. 1 illustrates an embodiment of a physiological measurement system 10 configured to determine a fractional oxygen saturation measurement. The system 10 includes a patient monitor 100 and optical sensor 200. The Monitor 100 includes a display screen 170, various user inputs 160 and sensor port 110. The sensor 200 includes emitters for emitting light of a plurality of wavelengths and a detector which detects light attenuated by tissue and generates a signal representative of the detected light. In operation, the patient monitor 100 sends drive signals to sensor 200 that turn the emitters on and off. The detector detects the light produced by the emitters after attenuation by the tissue. The detector generates a signal representative of the detected light and communicates the signal to the patient monitor 100. The patient monitor 100 includes a processor and other signal processing circuitry for processing the signal and determining physiological measurements from the signal. Greater detail of the patient measurement system 10 is disclosed in U.S. Pat. No. 7,764,982 entitled “Multiple Wavelength Sensor Emitters,” the contents of which is expressly incorporated herein in its entirety.

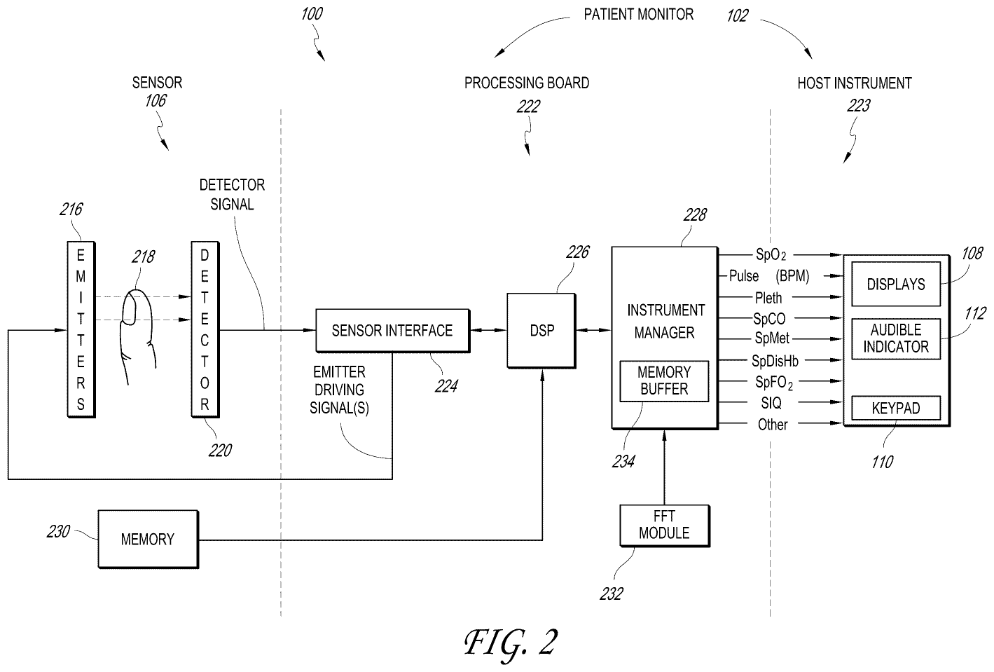

FIG. 2 illustrates details of an embodiment of a physiological measurement system 100 in a schematic form. Typically a sensor 106 includes energy emitters 216 located on one side of a patient monitoring site 218 and one or more detectors 220 located generally opposite. The patient monitoring site 218 is usually a patient's finger (as pictured), toe, ear lobe, or the like. Energy emitters 216, such as LEDs, emit particular wavelengths of energy through the flesh of a patient at the monitoring site 218, which attenuates the energy. The detector(s) 220 then detect the attenuated energy and send representative signals to the patient monitor 102. In an embodiment, 8 LEDs are used. In an embodiment 10 LEDs are used. In an embodiment, the LEDs have nominal wavelengths of about 620 nm, 630 nm, 650 nm, 660 nm, 730 nm, 805 nm, 905 nm, 1170 nm, 970 nm, 1270-1300 nm. Of course other nominal wavelengths in the same general range can be used as will be appreciated by a person of skill in the art.

In an embodiment, the patient monitor 102 includes processing board 222 and a host instrument 223. The processing board 222 includes a sensor interface 224, a digital signal processor (DSP) 226, and an instrument manager 228.

The host instrument typically includes one or more displays 108, control buttons 110, a speaker 112 for audio messages, and a wireless signal broadcaster. Control buttons 110 may comprise a keypad, a full keyboard, a track wheel, and the like. Additionally embodiments of a patient monitor 102 can include buttons, switches, toggles, check boxes, and the like implemented in software and actuated by a mouse, trackball, touch screen, or other input device.

The sensor interface 224 receives the signals from the sensor 106 detector(s) 220 and passes the signals to the DSP 226 for processing into representations of physiological parameters. These are then passed to the instrument manager 228, which may further process the parameters for display by the host instrument 223. In some embodiments, the DSP 226 also communicates with a memory 230 located on the sensor 106; such memory typically contains information related to the properties of the sensor that may be useful in processing the signals, such as, for example, emitter 216 energy wavelengths. The elements of processing board 222 provide processing of the sensor 106 signals. Tracking medical signals is difficult because the signals may include various anomalies that do not reflect an actual changing patient parameter. Strictly displaying raw signals or even translations of raw signals could lead to inaccurate readings or unwarranted alarm states. The processing board 222 processing generally helps to detect truly changing conditions from limited duration anomalies. The host instrument 223 then is able to display one or more physiological parameters according to instructions from the instrument manager 228, and caregivers can be more confident in the reliability of the readings. Among the various parameters that can be display are Sp0₂ (Oxygen Saturation), SpMet (Methemoglobin), SpCO (Carboxyhemoglobin), a combined dishemoglobin measurement (referred to herein as SpDisHb), and directly measured or derived Fractional Oxygen Saturation, referred to herein as SpF0₂ or SpFrac0₂ among other parameters including those illustrated in FIG. 2.

In an embodiment, a direct measurement of fractional oxygen saturation is obtained from the plethysmograph data using DSP 226. The measurement is based on empirically obtained data which is correlated with gold standard invasive measurements of fractional oxygen saturation which is used to obtain a calibration curve.

In another embodiment, a direct measurement of dishemoglobins are obtained. The dishemoglobin measurement is also obtained from the plethysmograph data using DSP 226. The measurement is based on empirically obtained data which is correlated with gold standard invasive measurements of dishemoglobins which is used to obtain a calibration curve. The dishemoglobin measurement is then used in conjunction with the oxygen saturation measurement as described below in order to obtain a fraction oxygen saturation measurement.

In yet another embodiment, a direct measurement of carboxyhemoglobin and methemoglobin is obtained and used to determine a fractional oxygen saturation measurement as described below.

In an embodiment, two or more of the above described processes for obtaining a fractional oxygen saturation measurement are used at the same time in order to obtain a more accurate measurement of oxygen saturation. For example, the various fractional oxygen saturation measurements can be compared or averaged. In an embodiment, signal confidence is determined for each measurement and used to determine which measurement determination is the best determination to use. In an embodiment, when signal confidence is low, the carboxyhemoglobin and methemoglobin measurements are used to determine a fraction saturation measurement. In an embodiment, when the signal confidence is high, a direct measurement of either the dishemoglobin or of the fraction oxygen saturation itself is used.

In an embodiment, the determination of fractional oxygen saturation begins with the understanding that hemoglobin in the blood falls into one of three categories. The three categories are: oxygenated hemoglobin, deoxygenated hemoglobin and dishemoglobins. Dishemoglobins include, for example, methemoglobin and carboxyhemoglobin. There may be additional categories of hemoglobin depending on patient conditions and inhaled toxins, however, for purposes of this fractional saturation measurement, it is assumed that these conditions are rare and/or negligible and are accounted for in the dishemoglobin measurement. Next, it is assumed that the relative fraction measured of these three categories of hemoglobin will add up to unity. This can be expressed mathematically as follows:

$$fRHb + fO 2 Hb + fDisHb = 1 \qquad 1$$

where fRHb, fO2Hb and fDisHb represent the fraction of the total amount of available hemoglobin that is in each hemoglobin state, deoxygenated hemoblogin, oxygenated hemoglobin and dishemoglobins. In an embodiment, the dishemolgobin measurement can be mathematically represented as:

$$fDisHb = fMetHb + fCOHb \qquad 2$$

Typical pulse oximeters measure an oxygen saturation measurement that is referred to as a functional oxygen saturation measurement or SaO2. Functional oxygen saturation is the percentage of oxygenated blood compared to total potential oxygen carrying capacity of the combined oxygenated and deoxygenated hemoglobin species. Functional oxygen saturation can be mathematically represented as follows:

$$SaO2 ( \% ) = \frac{{fO}_{2} Hb}{fRHb + {fO}_{2} Hb} * 100 \% \qquad 3$$

True fractional oxygen saturation or SpFO₂, as defined herein, provides a measure of oxygenated hemoglobin compared to the total of all hemoglobin in the blood. This includes oxygenated hemoglobin and deoxygenated hemoglobin as well as dishemoglobins (including methemoglobin and carboxyhemoglobin, for example). In an embodiment, fraction oxygen saturation is measured directly from the plethysmograph. Fractional oxygen saturation can also be mathematically represented as follows:

$$SpFO 2 ( \% ) = \frac{{fO}_{2} Hb}{fRHb + {fO}_{2} Hb + fDisHb} * 100 \% \qquad 4$$

Fractional oxygen saturation can be measured directly or derived from measureable parameters. As discussed above, typical pulse oximeters measure functional oxygen saturation. Some physiological measurement devices sold and marketed under the Rainbow® mark by Masimo Corp. of Irvine, CA are capable of measuring methemoglobin and carboxyhemoglobin in addition to functional oxygen saturation. Additionally, the dishemoglobins can be measured directly together as a single parameter combining methemoglobin and carboxyhemglobin as well as other dishemoglobins in order to avoid anomalies introduced by separate measurements. As described below, fractional oxygen saturation can be determined using measurements of SpO2, SpMet and SpCO. The following equations separate carboxyhemglobin and methemoglobin measurements, however, it is to be understood that the carboxyhemglobin and methemoglobin measurements need not be separate measurements but can be substituted for a single separate dishemoglobin measurement. Thus, for example, in equation 4 below, fMetHb+fCOHb can be substituted out for a single fDisHb (fractional dishemoglobin) measurement.

Equation 1 above can be mathematically rewritten as follows using a separate methemoglobin measurement and carboxyhemoglobin measurement instead of a single dishemoglobin measurement:

$$fRHb + {fO}_{2} Hb = 1 - fMetHb - fCOHb \qquad 5$$

Equation 3 above can also be mathematically rewritten as follows so that the left side of the equation matches the left side of equation 4:

$$fRHb + {fO}_{2} Hb = \frac{{fO}_{2} Hb}{SaO 2} * 100 \% \qquad 6$$

Equations 4 and 5 can be combined as follows:

$$\frac{{fO}_{2} Hb}{SaO 2} * 100 = 1 - fMetHb - fCOHb \qquad 7$$

Equation 6 can then be mathematically rewritten as follows:

$${fO}_{2} Hb = \frac{( 1 - fMetHb - fCOHb ) * SaO 2}{100} \qquad 8$$

Equation 7 can be multiplied by 100 to express fO2Hb, fMetHb, and fCOHb as a percentage, leading to the final equation for fractional oxygen saturation:

$${O}_{2} Hb = \frac{( 100 - MetHb - COHb ) * SaO 2}{100} \qquad 9$$

Finally, rewriting Equation 8 in terms of measureable parameters using a Masimo Rainbow patient monitor provides the following equation:

$${SpFO}_{2} = \frac{( 100 - SpMet - SpCO ) * SpO2\_Robust}{100} \qquad 10$$

Equation 9 above thus provides a calculated fractional saturation measurement that will give a patient care provider a more accurate indication of the physiological state of the patient.

One issue that arises with equation 9 is the accuracy of the various measurements involved. In particular, if one measurement is inaccurate, the accuracy of the fractional oxygen saturation measurement can be significantly affected. In order to minimize error in the fractional oxygen saturation measurement, the accuracy of the various measurements that form the fractional oxygen saturation measurement are determined. If one or more of the measurements are considered unreliable or inaccurate, weights or adjustments can be added in order to increase the reliability of the fractional oxygen saturation measurement.

In one embodiment, a rule based system is provided which analyzes the measurements based on various rules and adjusts parameter values in accordance with those rules. For example, if one parameter has a low confidence it can be down weighted in the determination of fractional oxygen saturation. Alternatively a confidence measure can be derived from the fractional oxygen measurement based on the confidence measure of SpO2, SpMet and SpCO. Many specific rules can be implanted to account for inaccuracies in the measurement.

In one embodiment, for example, high methemoglobin levels can impact the measurements of both carboxyhemoglobin and oxygen saturation. Thus, the present disclosure provides a rule based system for accounting for potential measurement inaccuracies during certain conditions.

In an embodiment, a more robust measurement of oxygen saturation is used. This measurement, referred to herein as Sp02_Robust, is determined using 2 or more wavelengths and a higher order polynomial fit. In an embodiment, 3 or more wavelengths can be used. In an embodiment, 5 wavelengths and a 3rd order polynomial equation are used to determine a more robust oxygen saturation measurement. Thus, equation 9 can be modified to:

$${SpFO}_{2} = \frac{( 100 - SpMet - SpCO ) * SpO2\_Robust}{100} \qquad 10$$

In an embodiment, a correction to SpCO is provided. As methemoglobin levels increase, the measurement of SpCO can be affected. In order to compensate for this, the following set of rules can be used. If the SpMet measurement is greater than a critical threshold, for example, about 2.2%, then the SpCO measurement is considered unreliable. This is referred to throughout this disclosure as a high methemoglobin condition. In this situation, SpCO is set to a relatively average SpCO measurement. In an embodiment, this can be the population average. In an embodiment, SpCO is set to about 0.9%.

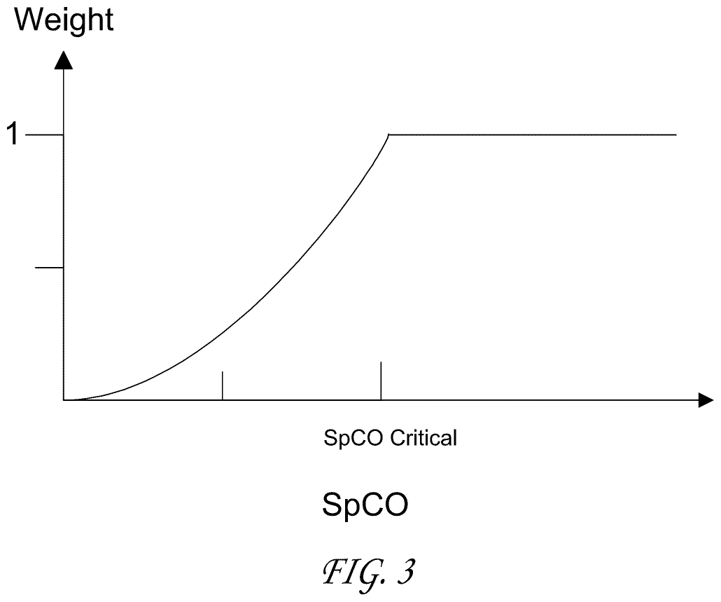

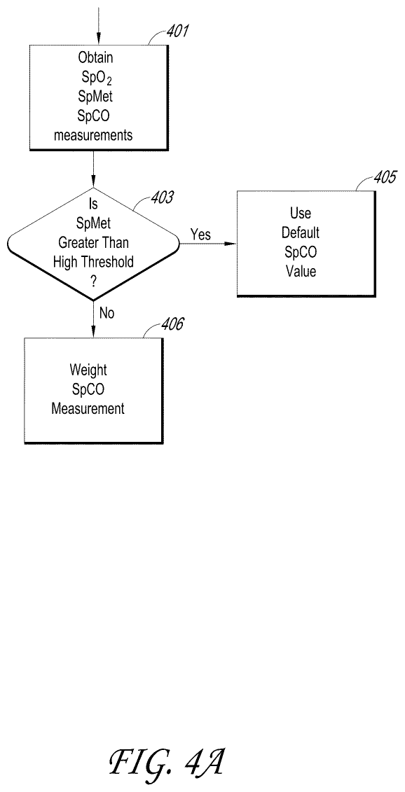

In one embodiment, if the SpMet measurement is below the high methemoglobin measurement threshold, then the SpCO measurement is weighted. This is referred to throughout this disclosure as a low methemoglobin condition. In an embodiment, the weighting provided to the SpCO measurement is directly related to the SpCO measurement. By way of example, the weighting scale depicted in FIG. 3 illustrates an embodiment of a weighting scheme for SpCO values. As depicted in FIG. 3, the greater the SpCO measurement, the greater the weight provided. In an embodiment, the SpCO critical value is about 7% to about 8%. Other weighting schemes can also be used. For example, a purely linear, exponential, partially linear and partially non-linear weighting scales could be used as well. In an embodiment, the SpCO critical value can be higher or lower.

In one embodiment, if the SpMet measurement is less than a low methemoglobin measurement threshold, then the SpCO measurement is weighted as discussed above. In an embodiment, a low SpMet measurement is below about 1%. If the SpMet measurement is between a low methemoglobin value and a high methemoglobin value, then the measured parameters are used without adjustment.

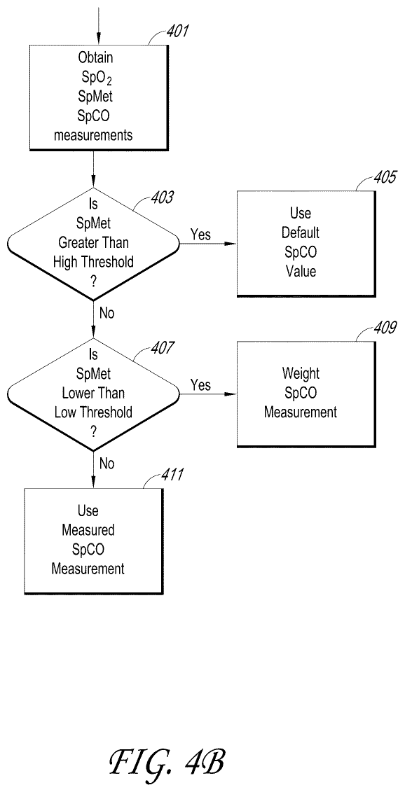

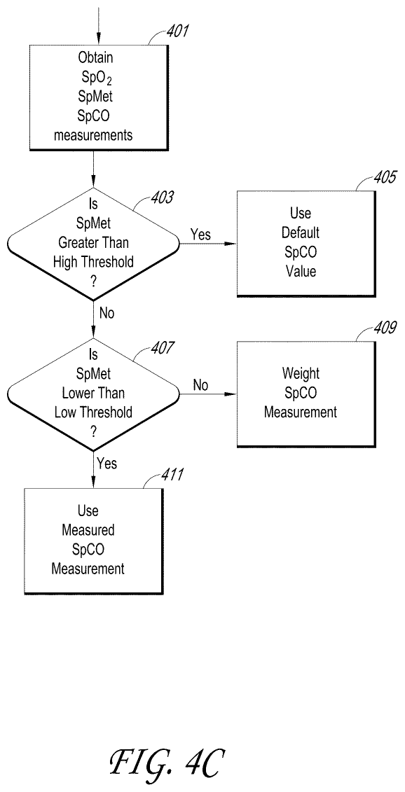

FIG. 4A illustrates an embodiment of a flow chart for determining whether a carboxyhemoglobin measurement needs to be adjusted. At step 401, oxygen saturation, carboxyhemoglobin and methemoglobin measurements are obtained. In an embodiment, the oxygen saturation measurement is a robust oxygen saturation measurement. The process moves to step 403 where a determination is made as to whether the methemoglobin measurement is greater than a threshold. If the answer is yes, the at step 405, a default carboxyhemglobin measurement value is used. If, at step 403, the answer is no, the process moves to step 406 where the carboxyhemoglobin measurement value is weighted as described above.

FIG. 4B illustrates another embodiment of a flow chart for determining whether a carboxyhemoglobin measurement needs to be adjusted. At step 401, oxygen saturation, carboxyhemoglobin and methemoglobin measurements are obtained. In an embodiment, the oxygen saturation measurement is a robust oxygen saturation measurement. The process moves to step 403 where a determination is made as to whether the methemoglobin measurement is greater than a threshold. If the answer is yes, the at step 405, a default carboxyhemglobin measurement value is used. If, at step 403, the answer is no, the process moves to step 407. At step 407, the determination is made as to whether the methemoglobin measurement is lower than a low threshold. If the answer is yes, then the carboxyhemoglobin measurement value is weighted as described above. If the answer is no at step 407, the process moves onto step 411 where the measured carboxyhemoglobin value is used in the above described process for determining a fractional oxygen saturation.

FIG. 4C illustrates another embodiment of a flow chart for determining whether a carboxyhemoglobin measurement needs to be adjusted. At step 401, oxygen saturation, carboxyhemoglobin and methemoglobin measurements are obtained. In an embodiment, the oxygen saturation measurement is a robust oxygen saturation measurement. The process moves to step 403 where a determination is made as to whether the methemoglobin measurement is greater than a threshold. If the answer is yes, the at step 405, a default carboxyhemglobin measurement value is used. If, at step 403, the answer is no, the process moves to step 407. At step 407, the determination is made as to whether the methemoglobin measurement is lower than a low threshold. If the answer is no, then the carboxyhemoglobin measurement value is weighted as described above. If the answer is yes at step 407, the process moves onto step 411 where the measured carboxyhemoglobin value is used in the above described process for determining a fractional oxygen saturation.

In an embodiment, a multivariate classifier is used to determine whether the SpMet and SpCO values should be categorized as either a high or low methemoglobin condition. The multivariate classifier determines clusters of classifications based on empirically obtained measurement data. The measurement under analysis is then compared with the multivariate classification information in order to determine into which classification, or cluster, the measurement should be placed. Once the measurement under analysis is classified into either a high or low methemoglobin condition, then the actions described above with respect to the high and low methemoglobin conditions respectively are taken.

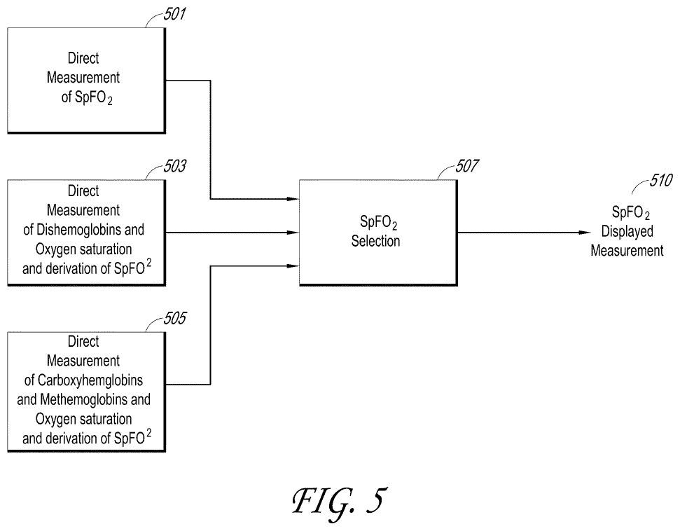

FIG. 5 illustrates an embodiment of a system that calculates a plurality of fractional oxygen saturation measurements and then determines which of the measurements to use in a final measurement. As illustrated in FIG. 5, a direct measurement of fractional oxygen saturation is determined at module 501. At module 503, a derived measurement fractional oxygen saturation is determined from a measurement of oxygen saturation and a direct dishemoglobin measurement. At module 505, a derived measurement fractional oxygen saturation is determined from a measurement of carboxyhemoglobin and methemoglobin measurements as described above. SpFO₂ determination module 507 then determines a final fraction oxygen saturation measurement 510. In an embodiment, the determination module looks at a signal confidence value to determine which measurement to use. If the signal confidence is low, the determination module 507 will use one or both of the output of modules 503 and 505. If the signal confidence is high, the determination module 507 will use the output of module 501. In an embodiment, the determination module 507 is configured to use an average or weighted average of all three measurements. In an embodiment, only two measurements are used. In an embodiment, more than three different determinations of fraction oxygen saturation are used and the determination modules 507 then determines an appropriate output.

Although the foregoing has been described in terms of certain specific embodiments, other embodiments will be apparent to those of ordinary skill in the art from the disclosure herein. Moreover, the described embodiments have been presented by way of example only, and are not intended to limit the scope of the disclosure. Indeed, the novel methods and systems described herein may be embodied in a variety of other forms without departing from the spirit thereof. Accordingly, other combinations, omissions, substitutions, and modifications will be apparent to the skilled artisan in view of the disclosure herein. Thus, the present disclosure is not limited by the preferred embodiments, but is defined by reference to the appended claims. The accompanying claims and their equivalents are intended to cover forms or modifications as would fall within the scope and spirit of the disclosure.

The methods, steps, processes, calculations, computations or the like (“methods”) provided herein are simplified examples that are generally performed by advanced processing devices, including complex signal processors, sensitive analog and digital signal preprocessing boards, optical/optoelectronic componentry, display drivers and devices, or similar electronic devices. An artisan will recognize from the disclosure herein that the various methods often must be performed at speeds that, as a practical matter, could never be performed entirely in a human mind. Rather, for many calculations providing real time or near real time solutions, outputs, measurements, criteria, estimates, display indicia, or the like, many of the foregoing processing devices perform tens to billions or more calculations per second. In addition, such processing devices may process electrical signals, infrared signals, wireless signals, or other electro-magnetic wave signals that are incomprehensible to a human mind in their raw form and at the speeds communicated.

1-19. (canceled)

20. A patient monitoring apparatus comprising:
a housing;
a memory device configured to store a signal generated by a noninvasive physiological sensor that monitors a patient, the signal being responsive to a physiological condition of the patient; and
one or more hardware processors in communication with the memory device, the one or more hardware processors being configured to:
determine a methemoglobin measurement and a carboxyhemoglobin measurement from the signal,
adjust the carboxyhemoglobin measurement based on the methemoglobin measurement to obtain an adjusted carboxyhemoglobin measurement, and
output for presentation an indicator responsive to the adjusted carboxyhemoglobin measurement.

21. The patient monitoring apparatus of claim 20, wherein the indicator represents a value of the adjusted carboxyhemoglobin measurement.

22. The patient monitoring apparatus of claim 20, wherein the one or more hardware processors are configured to adjust the carboxyhemoglobin measurement based on the methemoglobin measurement by setting the adjusted carboxyhemoglobin measurement to be a default carboxyhemoglobin measurement based on the methemoglobin measurement.

23. The patient monitoring apparatus of claim 20, wherein the one or more hardware processors are configured to adjust the carboxyhemoglobin measurement based on the methemoglobin measurement by scaling the carboxyhemoglobin measurement based on the methemoglobin measurement to obtain the adjusted carboxyhemoglobin measurement.

24. The patient monitoring apparatus of claim 20, wherein the one or more hardware processors are configured to determine a fractional oxygen saturation measurement from the adjusted carboxyhemoglobin measurement.

25. The patient monitoring apparatus of claim 24, wherein the one or more hardware processors are configured to determine the fractional oxygen saturation measurement further from the methemoglobin measurement.

26. The patient monitoring apparatus of claim 24, wherein the indicator represents a value of the fractional oxygen saturation measurement.

27. The patient monitoring apparatus of claim 20, wherein the signal is representative of light of three or more wavelengths that is detected after attenuation by tissue of the patient, and the one or more hardware processors are configured to determine the carboxyhemoglobin measurement from the light of three or more wavelengths that is detected after attenuation.

28. The patient monitoring apparatus of claim 20, wherein the signal is representative of light of five or more wavelengths that is detected after attenuation by tissue of the patient, and the one or more hardware processors are configured to determine the carboxyhemoglobin measurement from the light of five or more wavelengths that is detected after attenuation.

29. The patient monitoring apparatus of claim 20, further comprising a display configured to present the indicator.

30. A patient monitoring method comprising:
storing, by a memory device, a signal generated by a noninvasive physiological sensor monitoring a patient, the signal being responsive to a physiological condition of the patient;
determining, by one or more hardware processors, a methemoglobin measurement and a carboxyhemoglobin measurement from the signal;
adjusting, by the one or more hardware processors, the carboxyhemoglobin measurement based on the methemoglobin measurement to obtain an adjusted carboxyhemoglobin measurement; and
outputting, by the one or more hardware processors, for presentation an indicator responsive to the adjusted carboxyhemoglobin measurement.

31. The patient monitoring method of claim 30, wherein the indicator represents a value of the adjusted carboxyhemoglobin measurement.

32. The patient monitoring method of claim 30, wherein said adjusting the carboxyhemoglobin measurement based on the methemoglobin measurement comprises setting the adjusted carboxyhemoglobin measurement to be a default carboxyhemoglobin measurement based on the methemoglobin measurement.

33. The patient monitoring method of claim 30, wherein said adjusting the carboxyhemoglobin measurement based on the methemoglobin measurement comprises scaling the carboxyhemoglobin measurement based on the methemoglobin measurement to obtain the adjusted carboxyhemoglobin measurement.

34. The patient monitoring method of claim 30, further comprising determining, by the one or more hardware processors, a fractional oxygen saturation measurement from the adjusted carboxyhemoglobin measurement.

35. The patient monitoring method of claim 34, wherein said determining the fractional oxygen saturation measurement comprises determining the fractional oxygen saturation measurement further from the methemoglobin measurement.

36. The patient monitoring method of claim 34, wherein the indicator represents a value of the fractional oxygen saturation measurement.

37. The patient monitoring method of claim 30, wherein the signal is representative of light of three or more wavelengths that is detected after attenuation by tissue of the patient, and the carboxyhemoglobin measurement is determined from the light of three or more wavelengths that is detected after attenuation.

38. The patient monitoring method of claim 30, wherein the signal is representative of light of five or more wavelengths that is detected after attenuation by tissue of the patient, and the carboxyhemoglobin measurement is determined from the light of five or more wavelengths that is detected after attenuation.

39. The patient monitoring method of claim 30, further comprising presenting the indicator on a display.
